(19) United States
(12) Patent Application Publication
(10) Pub. No.: US 2025/0303024 A1
(43) Pub. Date: Oct. 2, 2025

(54) INDIVIDUALIZED NERVE CONDUIT WITH DIFFERENTIATED INTERFACES

(71) Applicant: SHANGHAI SIXTH PEOPLE'S HOSPITAL, Shanghai (CN)

(72) Inventors: Yun QIAN, Shanghai (CN); Lingchi KONG, Shanghai (CN); Cunyi FAN, Shanghai (CN); Zhengwei YOU, Shanghai (CN); Wei SUN, Shanghai (CN); Xin GAO, Shanghai (CN); Zhiwen YAN, Shanghai (CN)

(21) Appl. No.: 19/182,653

(22) Filed: Apr. 18, 2025

Related U.S. Application Data
(63) Continuation of application No. PCT/CN2023/083644, filed on Mar. 24, 2023.

(30) Foreign Application Priority Data
Mar. 3, 2023 (CN) 202310198696.2

(51) Int. Cl. A61L 27/24; A61L 27/18; A61L 27/20; A61L 27/22

(52) U.S. Cl. CPC A61L 27/24; A61L 27/18; A61L 27/20; A61L 27/222; A61L 27/225

(57) ABSTRACT

Disclosed is an individualized nerve conduit with differentiated interfaces, including a hollow outer sheath and an inner core filler disposed inside the hollow outer sheath; wherein a surface of the hollow outer sheath is distributed with nano-scale micropores, and the inner core filler is a loose filler of micron-scale sheets with axially oriented arrangement. The structure of such nerve conduit satisfies the repair microenvironment with different needs, both internal and external, and realizes the important function of “internal affinition and external alienation, internal promotion and external resistance”. The conduit not only promotes directional myelination of nerve and directional ingrowth of axon, but also prevents neuroma formation.

### TECHNICAL FIELD

The present disclosure relates to the field of nerve conduit technology, specifically to a individualized nerve conduit with differentiated interfaces, which is used for the repair and regeneration of defective nerves.

### DESCRIPTION OF THE PRIOR ART

Peripheral nerve injuries are common conditions in the emergency and orthopedic department due to high-energy trauma, post-tumor resection, and chronic systemic diseases. According to the Seddon classification criteria (according to the severity of the injury), the peripheral nerve injuries can be divided into neurapraxia, axonotmesis, and neurotmesis. For tension-free nerve gaps, “end-to-end anastomosis” is mostly used in clinical practice in the absence of large tension. While for severe nerve defects (defects longer than 4 cm for adults), autograft is still the gold standard. However, this treatment has important defects such as donor restriction, size mismatch and donor site damage, and long-term follow-ups show that the functional recovery of patients after treatment is very limited, which greatly limits its wide clinical application. With the expansion of theories and the advancement of tissue engineering technology in the field of life sciences, the role of nerve conduit bridging in clinical treatment has been continuously improved. Various functionalized nerve conduits are expected to replace autograft by efficiently mimicking the structure and function of peripheral nerve tissues, and become the first choice for clinical treatment.

At present, there are some improved designs in the nerve conduit market at home and abroad, which can not only bridge nerve stumps and provide certain mechanical support for peripheral nerve regeneration, but also guide axons to extend to the distal end through auxiliary substance exchange or the addition of inducible bioactive factors to achieve directional regeneration of peripheral nerves. However, these inventive designs are mainly based on the basic neuroadaptability, biocompatibility and physiology of peripheral nerve regeneration, and the resulting clinical products have shortcomings such as inaccurate therapeutic effect and rapid degradation in vivo. In addition, the addition of bioactive factors has the risk of inducing gene mutations, tumorigenesis and teratogenesis. In contrast, the regulatory effect of physical stimulation is definite, stable, sustainable, and safe to be applied in vivo, and it can avoid the deficiency of biochemical agents while exerting its function, which has a broader application prospect. Among the many physical intervention factors, the topological microstructure (i.e., biomimetic concept) given to nerve grafts by mimicking the oriented structure of peripheral nerves has attracted extensive attention, where the axial-oriented micro-nano structure has been confirmed to have the effect of orientating the growth of axon, which is of great significance for long-distance peripheral nerve defects.

In the prior art, there are descriptions of nerve conduits (CN201810660151.8, Multi-channel peripheral nerve conduit and preparation method thereof: CN201511003594.2, Artificial nerve scaffold and preparation method and application thereof). In previous nerve conduit products or experimental studies, electrospinning technology or additive manufacturing was mostly used to construct hollow nerve conduits, where the fibers arranged in the orientation of the wall of conduit can induce the axial migration of cells, and the topological structures such as micro-nano grooves on the fiber surface can increase the adhesion area of cells on the inner surface of the conduit, aiming to establish and maintain the regenerative microenvironment at an early stage. However, this structure means that there will be more fibrous tissue on the outer surface of the regenerated nerve that will adhere to and envelope, which will hinder the nerve repair process and even cause neuroma, making it difficult for high-quality nerve regeneration. Therefore, the lack of nerve conduits with differentiated interfaces between inside and outside makes it difficult for nerves to be reconstructed on demand, which is a huge problem in the current nerve repair market.

### SUMMARY OF THE INVENTION

In view of the deficiencies of the prior art, an object of the present disclosure is to provide a individualized nerve conduit with differentiated interfaces.

The present disclosure is a individualized nerve conduit with differentiated interfaces, where the biomimetic nerve conduit with special shape is customized for the defect repair of multi-branch nerve by 3D printing technology combined with phase separation and reverse mould method. Based on the three-dimensional digital modeling of fluorescent labeling, sucrose is used as the printing raw material and is heated in situ to be caramelized to obtain printing ink, and after 3D printing the customized sugar mold of the desired shape, a mixed solution such as polycaprolactone (PCL) with good mechanical strength and relatively slow degradation is cast on the sugar mold, and then the “outer sheath” of the biomimetic nerve graft is prepared by phase separation and template leaching. The gradient freezing method was used to form an axially oriented lamellar “inner core filler” in the “outer sheath” of solutions such as silk fibroin with good biocompatibility and relatively rapid degradation to guide the growth of nerve axons.

An object of the present disclosure is realized through the following technical solution:

The disclosure provides a individualized nerve conduit with differentiated interfaces, including a hollow outer sheath and an inner core filler disposed inside the hollow outer sheath.

A surface of the hollow outer sheath is distributed with nano-scale micropores, and the inner core filler is a loose filler of micron-scale sheets with axially oriented arrangement.

The degradation rate of the hollow outer sheath is less than that of the inner core filler. Preferably, the pore size of the nano-scale micropores is 400-800 nm.

The present disclosure also provides a preparation method of a individualized nerve conduit with differentiated interfaces, including:

S1. printing a sucrose template: setting nerve defect parameters, and performing 3D printing with caramelized ink to obtain the sucrose template:

S2. preparing a hollow outer sheath of the nerve conduit: dissolving raw materials for preparing the outer sheath in a solvent to form a mixed solution a: then immersing the sucrose template in the mixed solution a and taking the sucrose template out after full infiltration: obtaining a sucrose template wrapped with the outer sheath on a surface of the sucrose template after the solvent completely volatilizing: then dissolving the sucrose template in the outer sheath; and then obtaining the hollow outer sheath after freeze-drying; and

S3. forming an inner core filler of the nerve conduit in situ: dissolving raw materials for preparing the inner core filler in water to form a mixed solution b; then placing the hollow outer sheath vertically in the mixed solution b and fully soaked: then performing gradient freeze-drying or electric field action to form a directional frozen structure; and then performing freeze-drying to remove ice crystals and obtaining the nerve conduit with differentiated interfaces.

Preferably, specific steps of the step S1 are:

S11. opening the GeSim software: filling a screw barrel with sucrose after a machine self-test is completed: selecting needle specification: installing printing parts; and clicking a “Lock” button to lock:

S12. setting a heating temperature for heating to obtain a printable caramelized ink after measuring a height of a print head:

S13. performing 3D printing to obtain the sucrose template after setting the nerve defect parameters for printing and printing conditions.

Preferably, in the step S12, the heating temperature is 150-170° C., and a heating time is 0.5-2 h:

in the step S13, the printing conditions include: the printing temperature is 140-150° C., and a printing speed is 1-5 mm/s.

Preferably, in the step S2, the raw material for preparing the outer sheath is a raw material with good casting mechanical strength and relatively slow degradation, specifically including PCL, PLGA, collagen, and decellularized nerve matrix.

The solvent is selected from at least one of water and an organic solvent.

Further preferably, the raw material for preparing the outer sheath is PCL or a mixture of PCL and other bioactive substances, and the other bioactive substances are selected from at least one of decellularized nerve matrix, collagen and PLGA: for example, a mixture of PCL and decellularized nerve matrix, and the mixing mass ratio is 4:0.5-2.

Preferably, the organic solvent includes at least one of hexafluoroisopropanol, toluene, methanol, acetone, carbon tetrachloride, and dimethyl sulfoxide (DMSO).

Preferably, in the mixed solution a, the concentration of the raw material for preparing the outer sheath is 3-8% (w/v), and more preferably the concentration is 5% (w/v).

Preferably, in the step S2, the step of dissolving the sucrose template are as follows: soaking the sucrose template wrapped with the outer sheath on the surface in deionized water to fully dissolve the sucrose template.

Preferably, in the step S3, the raw materials for preparing the inner core filler are raw materials with good biocompatibility and relatively rapid degradation, specifically including silk fibroin, decellularized nerve matrix, hyaluronic acid, gelatin, sodium alginate.

Further preferably, the raw material for the preparation of the inner core filler is a mixture of silk fibroin and at least one of hyaluronic acid, gelatin and decellularized nerve matrix. For example, the raw material for the preparation of the inner core filler is a mixture of silk fibroin, hyaluronic acid and decellularized nerve matrix, and the mass ratio is 2:2-6:1, and the mass percentage content of hyaluronic acid in the obtained mixed solution b is 2-6%.

Preferably, in the S3, a temperature for soaking is 3-5° C., and a time for soaking is 2-4 h.

Preferably, in the step S3, the specific steps of gradient freeze-drying are as follows: pre-cooling the hollow outer sheath containing mixed solution b in the inner core to 0° C., and then placing the hollow outer sheath on a cold table at −190±10° C. for 20-40 minutes to form a directional frozen structure.

Preferably, in the specific steps of the gradient freeze-drying, the hollow outer sheath is placed on the cold table at −190° C. for 30 minutes.

The elastic modulus of nerve conduit of “inner core and outer sheath” with differentiated interfaces prepared according to the present disclosure ranges from 50-80 MPa.

Compared with the prior art, the beneficial effects of the present disclosure are:

(1) A nerve conduit with differentiated interfaces provided according to the present disclosure uses base materials that have been approved by the FDA to construct differentiated functional interfaces of “inner core and outer sheath”. The outer surface has strong anti-adhesion characteristics to prevent the invasion of fibrous tissue, and the nano-scale micropores which are uniformly distributed under the electron microscope are used for molecular exchange. The inner surface is a loose filler of micron-scale sheet arranged axially oriented. This structure satisfies the repair microenvironment with different needs, both internal and external, and realizes the important function of “internal affinition and external alienation, internal promotion and external resistance”. The conduit not only promotes directional myelination of nerve and directional ingrowth of axon, but also prevents neuroma formation.

(2) The present disclosure relies on the results obtained on the basis of previous research, and provides a nerve conduit with the most suitable topological structure parameters, where an axial arrangement structure suitable for the stretching and migration of cells such as vascular endothelial cells and neurons under the gradient freeze-drying or the action of electric field is formed. This structure has been experimentally confirmed to be conducive to guiding the rapid and efficient reconstruction of small blood vessels and remyelination after injury, and plays a “paving stone” role for the extension of axon stump.

(3) The present disclosure uses digital modeling combined with template leaching method to provide a individualized nerve conduit. The preparation process of the nerve conduit is convenient, and the product parameters are adjustable and controllable, which provides a reliable personalized treatment plan for nerve defects with complex structures such as multiple branches, and fills the gap of the lack of precise repair products for complex nerve defects in clinical practice.

### BRIEF DESCRIPTION OF DRAWINGS

Other features, purposes and advantages of the present disclosure will become more apparent by reading the detailed description of the non-restrictive embodiments with reference to the following drawings:

FIG. 1A is a schematic diagram of the surface microtopology of the “outer sheath” of the nerve conduit with differentiated interfaces prepared according to Embodiment 1:

FIG. 1B is a scanning electron microscope image of the “outer sheath” of the nerve conduit with differentiated interfaces prepared according to Embodiment 1:

FIG. 2A is a physical image of the cross-sectional ultra-depth of field of the “inner core filler” of the nerve conduit with differentiated interfaces prepared according to Embodiment 1:

FIG. 2B is an image of scanning electron microscope of the “inner core filler” of the nerve conduit with differentiated interfaces prepared according to Embodiment 1:

FIG. 2C is an image of bottom cross-sectional scanning electron microscope of the “inner core filler” of the nerve conduit with differentiated interfaces prepared according to Embodiment 1:

FIG. 3 is a schematic diagram of the cell-core interface during the repair of the nerve conduit of “inner core and outer sheath”:

FIG. 4A is a physical image of the nerve conduit with differentiated interfaces prepared according to Embodiment 1:

FIG. 4B is an image of implantation in vivo of the nerve conduit with differentiated interfaces prepared according to Embodiment 1:

FIG. 5A is a footprint pattern of a gait analysis result of rats after 12 weeks of implantation of the nerve conduits prepared according to Embodiment 1, Comparative example 1 and Comparative example 2 and autologous nerves of the rats:

FIG. 5B is a shape of the rat foot of a gait analysis result of rats after 12 weeks of implantation of the nerve conduits prepared according to Embodiment 1, Comparative example 1 and Comparative example 2 and autologous nerves of the rats:

FIG. 5C is a sciatic nerve function index (SFI) analysis of a gait analysis result of rats after 12 weeks of implantation of the nerve conduits prepared according to Embodiment 1, Comparative example 1 and Comparative example 2 and autologous nerves of the rats:

FIG. 6 is a transmission electron microscope (TEM) image at different magnifications of the regenerated nerve after 12 weeks of implantation of the nerve conduits prepared according to Embodiment 1, Comparative example 1 and Comparative example 2 and after 12 weeks of autograft:

FIG. 7 is the results of Masson staining and HE staining of rat gastrocnemius muscle after 12 weeks of implantation of the nerve conduits prepared according to Embodiment 1, Comparative example 1 and Comparative example 2 and after 12 weeks of autograft:

FIG. 8 is the in vitro experimental results of the inner core filler prepared according to Embodiment 1 and Comparative example 2 on the migration of Schwann cells; and

FIG. 9 is a schematic diagram of the structure of the “inner core and outer sheath” multi-branch nerve conduit.

### DESCRIPTION OF EMBODIMENTS

The present disclosure is described in detail below in conjunction with specific embodiments. The following embodiments will help those skilled in the art to further understand the present disclosure, but do not limit the disclosure in any form. It should be noted that for a person of ordinary skill in the art, a number of modifications and improvements can be made without departing from the conception of the present disclosure. These are within the scope of protection of the present disclosure.

The words “preferred”, “preferably”, “more preferred”, etc. in the present disclosure refer to, in certain circumstances, embodiments of the present disclosure that may provide certain beneficial effects. However, in the same situation or in other situations, other embodiments may also be preferred. In addition, the formulation of one or more preferred embodiments does not imply that the other embodiments are unavailable, nor is it intended to exclude the other embodiments from the scope of the present disclosure.

It should be understood that, except in any example of operation, or in the case otherwise indicated, all figures representing, for example, the amount of the ingredients used in the description and claims shall be understood as being modified in all cases by the term “approximately”. Therefore, unless otherwise noted, the numerical parameters set forth in the following description and the accompanying claims are approximations that vary according to the desired performance to be obtained by the present disclosure. At least not an attempt to limit the application of the doctrine of equivalent to the claims, each numerical parameter should at least be interpreted in terms of the number of significant figures reported and by applying ordinary rounding techniques.

Although the range of values and parameters illustrating the wide range of the present disclosure are approximate, the numerical values listed in the specific examples are reported as accurately as possible. However, any numerical value inherently contains certain errors that necessarily arise from the standard deviations found in their respective test measurements.

In the previous research of the applicant's team, it was found that the nerve conduits prepared currently still has the following problems:

### 1) Lack of Internal and External Differentiated Interfaces

At present, most of the studies adopt the design of hollow structure in the conduit, and the wall of conduit is not permeable. The internal interface is disordered, which cannot achieve precise neurovascular repair. Then, in order to solve this problem, although the topological design of the oriented structure accelerates the speed of nerve repair, it brings the problem of increased adhesion on the outer surface of the conduit. Therefore, there is currently a lack of nerve conduits with differentiated functional interfaces between internal and external clinics. The main reason is that the required microenvironments inside and outside the conduit are quite different, and it is difficult for a single substrate or a single preparation process to achieve the optimal internal and external functional requirements at the same time.

In addition, according to the pathophysiological characteristics of nerve regeneration, the degradation rates required for the internal and external interfaces are different: the external interface, as a mechanical support and anti-adhesion physical barrier, needs to be degraded slowly, and the internal interface needs to be degraded synchronously on the basis of guiding nerve regeneration, otherwise a physical obstacle will be formed.

### 2) Unclear Biomimetic Parameters

The existing research and development of biomimetic products mainly focused on analyzing the natural anatomical structure or physiological characteristics of tissues, and then simulating them at the physical, chemical or biological levels. However, the applicant's team has recently found that the neural regeneration microenvironment is significantly different from the natural physiological characteristics, and the repair effect of simulating the post-injury regeneration microenvironment is better than that of natural structural biomimicry. Therefore, there is a lack of unified standards and consensus on the biomimetic concept of neural structure, and the optimal parameters of biomimetic nerve conduit products are not clear, which makes it difficult to prepare neural grafts with optimal parameters.

### 3) Difficulty in Achieving Individualized Nerve Repair

At present, the nerve conduits available for clinical use are all single-straight tubular products, which cannot meet the needs of defects of multiple branch nerve segments (such as the sciatic nerve at the bifurcation of the tibial nerve and the common peroneal nerve). In clinical practice, the above limitations often lead to atrophy of target organs, causing decreases in the quality of life of patients. Post-traumatic nerve defects vary in shape and size, which brings difficulties to personalized repair, and it is difficult for templated preparation processes or products to form advantages. Therefore, it is essential to develop individual product preparation methods.

Based on this, the applicant's team proposed for the first time the theory of “building structures with differentiated interfaces on demand”, in which the inner and outer surfaces of the conduit should conform to the requirements of different environments, and have the important functions of “internal affinition and external alienation, internal promotion and external resistance”. The present disclosure aims to construct a individualized biomimetic nerve conduit with differentiated functional interfaces, and to clarify its preparation method, which can be used to guide the efficient regeneration of blood vessels and nerves. According to the conduit of the present disclosure, after obtaining the defect parameters based on the individualized needs of nerve repair, the 3D print of a sugar mold is performed quickly and calibrated, and then the adapted outer sheath with anti-adhesion function is obtained using the template leaching method. An inner core with axial topology is formed in situ inside the outer sheath using the electric field directional guidance combined with freeze-drying. The shape, size, and branch angle of the nerve conduit can be customized, and the differentiated interface formed in situ is conducive to improving the repair efficiency of the nerve graft and accelerating the regeneration of peripheral nerves.

In the following embodiments, there are two reasons for selecting sucrose as printing ink: First, because sucrose has good fluidity, processability and formability in the molten state, and second, sucrose can quickly dissolve in water, which is a good sacrificial template.

The following embodiments specifically provide a preparation method of a individualized nerve conduit with differentiated interfaces, including:

S1. printing a sucrose template: setting nerve defect parameters, and performing 3D printing with caramelized ink to obtain the sucrose template:

S2. preparing a hollow outer sheath of the nerve conduit: dissolving raw materials for preparing the outer sheath in a solvent to form a mixed solution a: then immersing the sucrose template in the mixed solution a and taking the sucrose template out after full infiltration: obtaining a sucrose template wrapped with the outer sheath on a surface of the sucrose template after the solvent completely volatilizing: then dissolving the sucrose template in the outer sheath; and then obtaining the hollow outer sheath after freeze-drying:

S3. forming an inner core filler of the nerve conduit in situ: dissolving raw materials for preparing the inner core filler in water to form a mixed solution b: then placing the hollow outer sheath vertically in the mixed solution b and fully soaked: then performing gradient freeze-drying or electric field action to form a directional frozen structure; and then performing freeze-drying to remove ice crystals and obtaining the nerve conduit with differentiated interfaces.

In the following embodiments, the polycaprolactone (PCL) used is produced by Aldrich Company of USA, with Mn=80000 g/mol. The hyaluronic acid used is produced by Shanghai Macklin Biochemical Technology Co., Ltd., with a purity of 97%. The decellularized matrix (dnECM) used is self-made in the laboratory, and please refer to the following document for its specific formula and preparation method “Fangsong Zhang, et al. Decellularized nerve extracellular matrix/chitosan crosslinked by genipin to prepare a moldable nerve repair material. Cell Tissue Bank (2021) 22:419-430”.

### Embodiment 1

In this embodiment, a nerve conduit of “inner core and outer sheath” with differentiated interfaces applied to a 15 mm rat sciatic nerve defect model is provided, and the preparation steps of which are as follows:

(1) Determining that an area to be implanted is a nerve defect with a diameter of 2.5 mm and a length of 15 mm.

(2) Printing a sucrose template: opening the GeSim software: filling ¾ volume of a screw barrel with sucrose after the machine self-test completed: selecting a needle of 1.2 mm: installing printing parts; and clicking a “Lock” button to lock. A heating temperature is set to 160° C. to heat for 1 h to obtain a printable caramelized ink after measuring the height of a print head. The temperature is set to 145° C.: after the temperature drops and remains stable, the 3D printing parameters are set in the software “Scaffold”. “Height” is set to 2.0 mm, “Angle Change” is set to +90°, “Infill Distance” is set to 1.4 mm, “Speed” is set to 2.4 mm/s, and “Feed” is set to 4 m/s. The corresponding parameters are set according to the diameter of 2.5 mm and the length of 15 mm. Then printing is started to get the sucrose template.

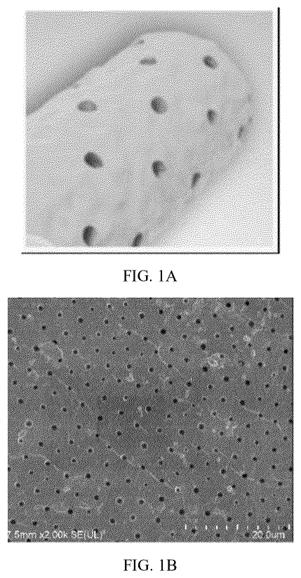

(3) Preparing an “outer sheath” of the nerve conduit: dissolving 0.4 g of poly caprolactone (PCL) and 0.1 g of decellularized matrix (dnECM) in 10 mL of hexafluoroisopropanol (HFIP), and performing magnetic stirring for 8 h at room temperature to form a colorless clear solution to obtain a PCL/dnECM mixed solution with a concentration of 5% (w/v). The sucrose template prepared in step (2) is immersed in the PCL/dnECM mixed solution prepared above for 10s-15s to ensure that the solution is fully infiltrated on the surface of the template; and then the sucrose template is clamped out with a tweezer to volatilize the solvent (hexafluoroisopropanol) for 10 minutes; and then infiltration and volatilization are repeated for 6 times. The sucrose template to which the PCL/dnECM mixed solution is attached after infiltration is taken out: after the solvent (hexafluoroisopropanol) is volatilized (being volatilized for 2 h), the sucrose template is soaked in deionized water for 2 h, during which the deionized water is replaced every 30 minutes to fully dissolve the sucrose template; and the remainder is taken out and lyophilized to obtain a hollow “outer sheath”. As shown in FIG. 1, where FIG. 1A is a schematic diagram of the microtopology of the “outer sheath”, and FIG. 1B is an image of scanning electron microscope. As can be seen in FIG. 1, the surface of the “outer sheath” has uniformly distributed nano-scale pores (pore size about 612.5±107 nm).

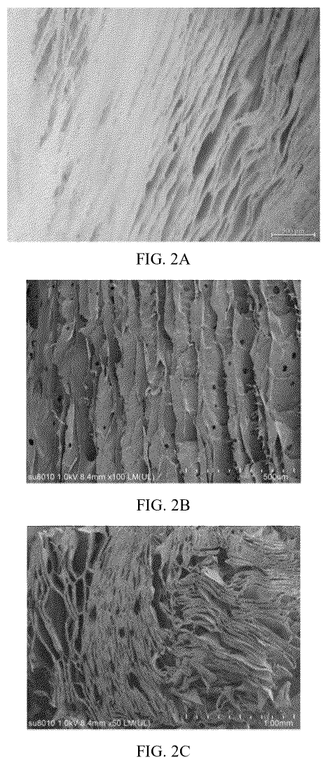

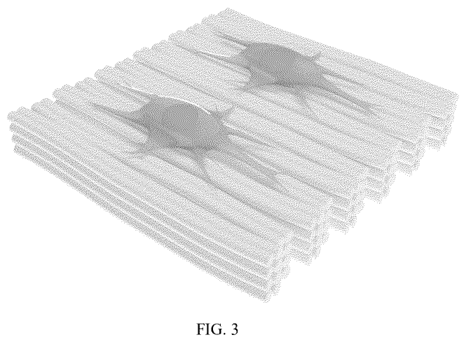

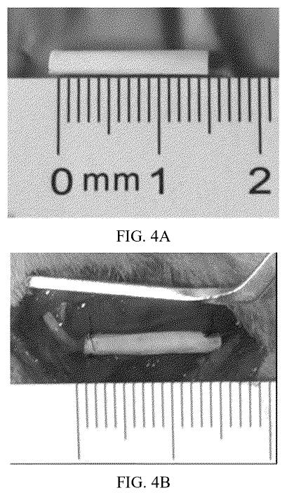

(4) Forming an “inner core” of the nerve conduit in situ: dissolving 0.02 g of decellularized matrix (dnECM), 0.04 g of silk fibroin (SF) and 0.04 g of hyaluronic acid (HA) in 2 mL of deionized water to form a colorless clear solution, to obtain a mixed aqueous solution of HA/dnECM/SF with a concentration of HA of 2% (w/v). The hollow “outer sheath” obtained from step (3) is placed vertically in a sample bottle filled with the above-mentioned mixed aqueous solution for “inner core filler”, and after fully soaked at 4° C. for 4 h, the sample bottle is pre-cooled to 0° C.; and then the sample bottle is placed on a cold table at −190° C. for 30 min to form a directional frozen structure; and then the ice crystals are removed by freeze-drying (24-36 h) to obtain a nerve conduit of “inner core and outer sheath” with differentiated interfaces with a diameter of 2.5 mm and a length of 15 mm (as shown in FIG. 2, FIG. 3 and FIG. 4). An physical image of the cross-sectional ultra-depth of field of the inner core filler of the obtained nerve conduit is shown in FIG. 2A, an image of cross-sectional scanning electron microscope is shown in FIG. 2B, and an image of bottom cross-sectional scanning electron microscope is shown in FIG. 2C. FIG. 3 illustrates an interface between cells and inner core fillers in the nerve conduit, showing that cells (including vascular endothelial cells, Schwann cells, and neuronal cells) are oriented to stretch and migrate on an axially oriented core structure to achieve rapid repair. FIG. 4A is a physical image of the prepared nerve conduit of “inner core and outer sheath” with differentiated interfaces. The test results show that its elastic modulus is 68.5±7.3 Mpa.

### Embodiment 2

The present embodiment provides a preparation method of a nerve conduit of “inner core and outer sheath” with differentiated interfaces, and the specific preparation steps of which are basically the same as those in Embodiment 1, except that: in the step (4), the addition amount of hyaluronic acid is 0.08 g, and a mixed aqueous solution of HA/dnECM/SF with an HA concentration of 4% (w/v) is obtained.

The structure of the finally prepared nerve conduit of “inner core and outer sheath” with differentiated interfaces is basically the same as Embodiment 1. The test results show that its elastic modulus is 66.4±8.1 Mpa.

### Embodiment 3

The present embodiment provides a preparation method of a nerve conduit of “inner core and outer sheath” with differentiated interfaces, and the specific preparation steps of which are basically the same as those in Embodiment 1, except that: in the step (4), the addition amount of hyaluronic acid is 0.12 g, and a mixed aqueous solution of HA/dnECM/SF with an HA concentration of 6% (w/v) is obtained.

The structure of the finally prepared nerve conduit of “inner core and outer sheath” with differentiated interfaces is basically the same as Embodiment 1. The test results show that its elastic modulus is 65.1±6.7 Mpa.

### Comparative Example 1

This Comparative example provides a hollow nerve conduit, i.e., the “outer sheath” of nerve conduit without the “inner core filler” described in Embodiment 1, and the preparation method thereof is the same as the steps (1)-(3) in Embodiment 1.

### Comparative Example 2

This Comparative example provides a nerve conduit, and the preparation method thereof is substantially the same as Embodiment 1. The difference is that forming an “inner core” of the nerve conduit in situ of the step (4) is dissolving 0.02 g of decellularized matrix (dnECM), 0.04 g of silk fibroin (SF) and 0.04 g of hyaluronic acid (HA) in 2 mL of deionized water to form a colorless clear solution, to obtain a mixed aqueous solution of HA/dnECM/SF with a concentration of HA of 2% (w/v). The hollow “outer sheath” obtained from step (3) is placed vertically in a sample bottle filled with the above-mentioned mixed aqueous solution for “inner core filler”, and after fully soaked at 4° C. for 4 h, the sample bottle is pre-cooled to 0° C.; and then the ice crystals are removed by freeze-drying (24-36 h) to obtain a nerve conduit of “inner core and outer sheath” with a diameter of 2.5 mm and a length of 15 mm. The inner core filler of this nerve conduit does not have an axially oriented arrangement, but rather a random arrangement.

### Effect Verification:

### 1. In Vivo Experiments

The nerve conduits prepared by the above Embodiment 1, Comparative example 1 and Comparative example 2 method are respectively implanted into the rat nerve defect (as shown in FIG. 4B), and the autologous nerves are transplanted into the rat nerve defect as a comparison.

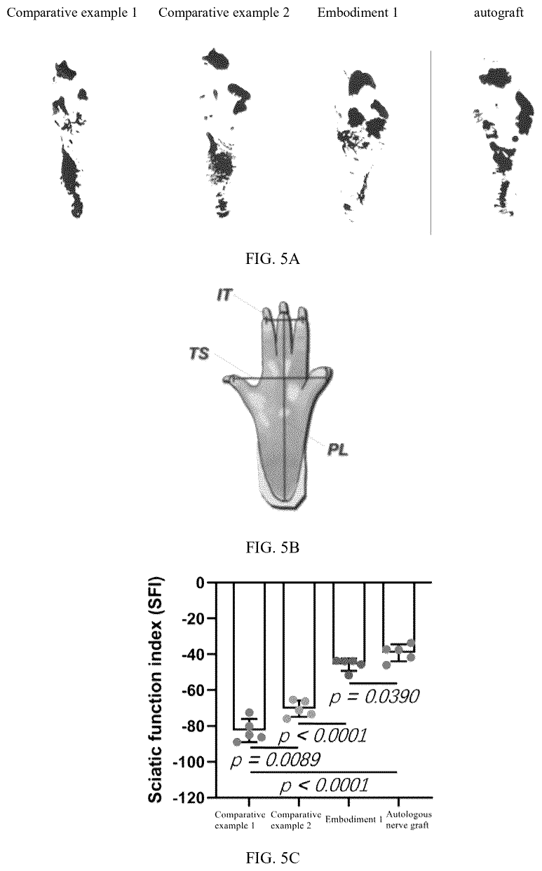

After 12 weeks, the gaits of the rats are analyzed, and the results are shown in FIG. 5, where FIG. 5A is the footprint morphology of rats in different experimental groups, FIG. 5B is the shape of rat feet, and FIG. 5C is the results of the analysis of sciatic nerve function index (SFI) of rats in each experimental group. According to the morphology of the footprints of rats in different experimental groups, the footprints of the group of rats in which the nerve conduit according to Embodiment 1 are implanted are similar to those of the group of rats of the autograft, while the footprints of the groups of rats in which the nerve conduit according to Comparative example 1 and Comparative example 2 are obviously long and narrow; and the toes are curled. The analyses and statistics are carried out on the footprints. The sciatic nerve function index (SFI) is calculated according to the distances of IT, TS and PL, with a range of −100 to 0, and the closer to 0) the better the neurological function (SFI is calculated as SFI=(−38.3×(EPL−NPL)/NPL)+(109.5×(ETS−NTS)/NTS)+(13.3×(EIT−NIT)/NIT) −8.8). The results of analysis of variance for each group in FIG. 5C show that the differences are statistically significant, and show that the neurological function of the group of Embodiment 1 is significantly better than that of the groups of Comparative example 1 and Comparative example 2.

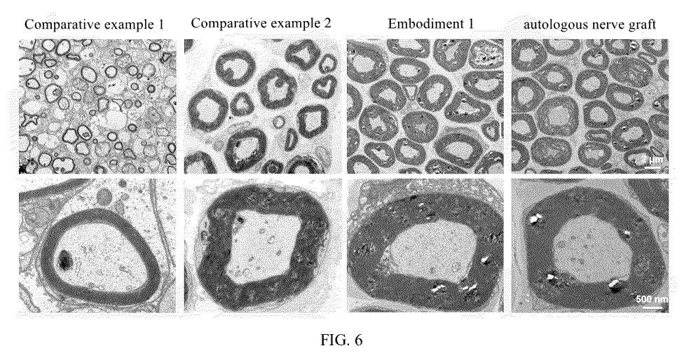

After 12 weeks, the quality of nerve regeneration is observed (as shown in FIG. 6). FIG. 6 is the transmission electron microscope (TEM) images at different magnifications of the regenerated nerves after 12 weeks of implantation of the nerve conduits prepared according to Embodiment 1, Comparative example 1 and Comparative example 2 and after 12 weeks of tautograft respectively. From the results of FIG. 6, it can be seen that the nerve conduit prepared by the Comparative example 1 has only a small amount of formed myelin sheath, and the myelin sheath thickness of the nerve conduit prepared by the Comparative example 2 has increased, while the myelin sheath thickness and axon diameter of the nerve conduit prepared by Embodiment 1 and the autograft are comparable and significantly better than the nerve conduits prepared by the Comparative example 1 and the Comparative example 2.

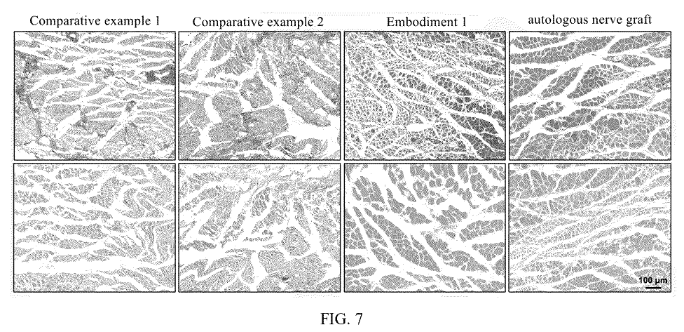

After 12 weeks, the gastrocnemius muscle is taken to explore the nutritional effect of nerve recovery on the innervated muscles. The results of Masson staining and HE staining of rats in each experimental group are shown in FIG. 7, which show that muscle fibers implanted with the groups of nerve conduit of Comparative example 1 and Comparative example 2 atrophy and have a large amount of collagen deposition (blue part), while the collagen deposition in the group implanted with nerve conduits of Embodiment 1 is significantly less than that in the groups of Comparative example 1 and Comparative example 2. Moreover, the muscle fiber density and muscle fiber diameter of the groups of nerve conduit of Embodiment 1 and the autograft are significantly better than those in the groups of nerve conduit of Comparative example 1 and Comparative example 2.

### 2. In Vitro Experiments

Vascular endothelial cells are implanted at different interfaces and then co-cultured with Schwann cells to observe the migration of Schwann cells. The specific experimental method is as follows: the transwell chambers with 8.0 μm pore size is placed in a 24-well plate, and the bottom surface of the lower chamber is laid with an orientated arrangement of the inner core (Embodiment 1), a scrambled arrangement of the inner core (Comparative example 1) or a blank control (tissue culture plate control); and then the vascular endothelial cells (HUVECs) are plated on the above different interfaces, and 1.5×104 Schwann cells (mSCs or RSC96) are inoculated in the upper chamber of the transwell chambers; the Schwann cells are fixed after 24 hours and the residual cells on the upper surface of the upper chamber are removed: the cells passing through the chamber are labeled by crystal violet staining and are photographed and counted to be analyzed.

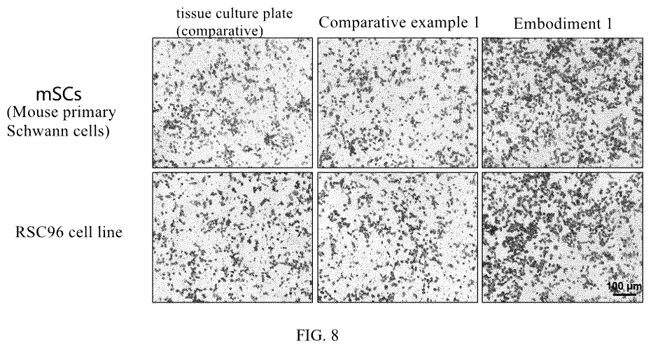

As shown in FIG. 8, the migration of Schwann cells is significantly promoted after co-culture of endothelial cells planted in the core filler with an orientated arrangement structure prepared in Embodiment 1 and Schwann cells surrounding neurons (the number of Schwann cells migrated in the figure increased).

It should be noted that, based on the specific preparation method shown in the embodiments of the present disclosure, the raw material for preparing the outer sheath can also be pure PCL or the outer sheath mixed solution prepared by PCL and decellularized nerve matrix in other mass ratios. The raw materials for the preparation of the inner core can also use the following different combinations to prepare the mixed solution of the inner core filler: (1) silk fibroin, hyaluronic acid (such as 1%, 2%, 4% of content): (2) silk fibroin, gelatin (such as 1%, 2%, 4% of content): (3) silk fibroin, decellularized nerve matrix, hyaluronic acid: (4) silk fibroin, decellularized nerve matrix, gelatin. From this, the nerve conduits with differentiated interfaces can be prepared, which has a hollow outer sheath with a surface distributed with nano-scale micropores and has a loose filler of micro-scale sheets with axially orientated arrangement as an inner core filler.

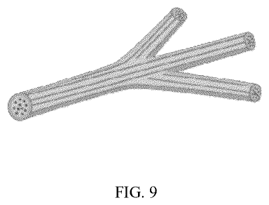

It should also be noted that through the parameter setting in 3D printing, the sucrose template of the multi-branch nerve conduit shown in FIG. 9 can be obtained accordingly, and the corresponding nerve conduit with multi-branch and differentiated interfaces can be prepared.

There are many specific ways of application of the present disclosure, and the above is only the preferred embodiment of the present disclosure. It should be noted that the above embodiments are only used to illustrate the present disclosure and are not used to limit the scope of protection of the present disclosure. It should be noted that for those of ordinary skill in the art, without departing from the principles of the present disclosure, certain improvements may also be made, and these improvements should also be regarded as the scope of protection of the present disclosure.

What is claimed is:

1. A individualized nerve conduit with differentiated interfaces, comprising a hollow outer sheath and an inner core filler disposed inside the hollow outer sheath:
wherein a surface of the hollow outer sheath is distributed with nano-scale micropores, and the inner core filler is a loose filler of micron-scale sheets with axially oriented arrangement:
the method for preparing the individualized nerve conduit with differentiated interfaces, comprising:
S1. printing a sucrose template: setting nerve defect parameters, and performing 3D printing with caramelized ink to obtain the sucrose template:
S2. preparing a hollow outer sheath of the nerve conduit: dissolving raw materials for preparing the outer sheath in a solvent to form a mixed solution a: then immersing the sucrose template in the mixed solution a and taking the sucrose template out after full infiltration: obtaining a sucrose template wrapped with the outer sheath on a surface of the sucrose template after the solvent completely volatilizing: then dissolving the sucrose template in the outer sheath; and then obtaining the hollow outer sheath after freeze-drying; and
S3. forming an inner core filler of the nerve conduit in situ: dissolving raw materials for preparing the inner core filler in water to form a mixed solution b: then placing the hollow outer sheath vertically in the mixed solution b and fully soaked: then performing gradient freeze-drying or electric field action to form a directional frozen structure; and then performing freeze-drying to remove ice crystals and obtaining the nerve conduit with differentiated interfaces.

2. The individualized nerve conduit with differentiated interfaces of claim 1, wherein specific steps of step S1 comprise:
S11. opening the GeSim software: filling a screw barrel with sucrose after a machine self-test is completed: selecting needle specification: installing printing parts; and clicking a “Lock” button to lock:
S12. setting a heating temperature for heating to obtain a printable caramelized ink after measuring a height of a print head:
S13. performing 3D printing to obtain the sucrose template after setting the nerve defect parameters for printing and printing conditions.

3. The individualized nerve conduit with differentiated interfaces of claim 2, wherein in the step S12, the heating temperature is 150-170° C., and a heating time is 0.5-2 h; and
in the step S13, the printing conditions comprise: the printing temperature is 140-150° C., and a printing speed is 1-5 mm/s.

4. The individualized nerve conduit with differentiated interfaces of claim 1, wherein in step S2, the raw materials for preparing the outer sheath comprise at least one of PCL, PLGA, collagen and decellularized nerve matrix:
the solvent is selected from at least one of water and an organic solvent.

5. The individualized nerve conduit with differentiated interfaces of claim 4, wherein the organic solvent comprises at least one of hexafluoroisopropanol, toluene, methanol, acetone, carbon tetrachloride, and dimethyl sulfoxide.

6. The individualized nerve conduit with differentiated interfaces of claim 1, wherein in the step S2, a step of dissolving the sucrose template is: soaking the sucrose template wrapped with the outer sheath on the surface in deionized water to fully dissolve the sucrose template.

7. The individualized nerve conduit with differentiated interfaces of claim 1, wherein in step S3, the raw materials for preparing the inner core filler comprise at least one of silk fibroin, decellularized nerve matrix, hyaluronic acid, gelatin, and sodium alginate.

8. The individualized nerve conduit with differentiated interfaces of claim 1, wherein in step S3, a temperature for soaking is 3-5° C., and a time for soaking is 2-4 h.

9. The individualized nerve conduit with differentiated interfaces of claim 1, wherein in the step S3, specific steps of the gradient freeze-drying are as follows: pre-cooling the hollow outer sheath containing mixed solution b in the inner core to 0° C., and then placing the hollow outer sheath on a cold table a −190±10° C. for 20-40 minutes to form a directional frozen structure.
